(19) United States
(12) Patent Application Publication
(10) Pub. No.: US 2020/0027530 A1
(43) Pub. Date: Jan. 23, 2020

(54) Simulating Patients for Developing Artificial Intelligence Based Medical Solutions

(71) Applicant: International Business Machines Corporation, Armonk, NY (US)

(72) Inventors: Lilla Boroczky, Mount Kisco, NY (US); Paul Dufort, Toronto (CA); Yiting Xie, Cambridge, MA (US); David Richmond, Newton, MA (US)

(21) Appl. No.: 16/185,874

(22) Filed: Nov. 9, 2018

Related U.S. Application Data
(63) Continuation-in-part of application No. 16/038,478, filed on Jul. 18, 2018.

(51) Int. Cl. G16H 10/20; G16H 10/40; G16H 30/40; G06N 3/02

(52) U.S. Cl. CPC G16H 10/20; G16H 10/40; G16H 50/70; G06N 3/02; G16H 30/40

(57) ABSTRACT

Mechanisms are provided to implement a cognitive artificial intelligence training mechanism for simulating patients for developing artificial intelligence based medical solutions. The cognitive artificial intelligence training mechanism perturbs non-image based information of a real patient from a real patient data set forming perturbed non-image based information, The cognitive artificial intelligence training mechanism generates an artificial patient data in an artificial patient data set using the perturbed non-image based information and a non-perturbed medical image of the real patient. The cognitive artificial intelligence training mechanism then trains an operation of a learning algorithm utilized by the cognitive data processing system using real patient data in the real patient data set and the artificial patient data in the artificial patient data set.

### BACKGROUND

The present application relates generally to an improved data processing apparatus and method and more specifically to mechanisms for simulating patients for developing artificial intelligence based medical solutions.

Decision-support systems exist in many different industries where human experts require assistance in retrieving and analyzing information. An example that will be used throughout this application is a diagnosis system employed in the healthcare industry. Diagnosis systems can be classified into systems that use structured knowledge, systems that use unstructured knowledge, and systems that use clinical decision formulas, rules, trees, or algorithms. The earliest diagnosis systems used structured knowledge or classical, manually constructed knowledge bases. The Internist-I system developed in the 1970s uses disease-finding relations and disease-disease relations. The MYCIN system for diagnosing infectious diseases, also developed in the 1970s, uses structured knowledge in the form of production rules, stating that if certain facts are true, then one can conclude certain other facts with a given certainty factor. DXplain, developed starting in the 1980s, uses structured knowledge similar to that of Internist-I, but adds a hierarchical lexicon of findings.

Iliad, developed starting in the 1990s, adds more sophisticated probabilistic reasoning where each disease has an associated a priori probability of the disease (in the population for which Iliad was designed), and a list of findings along with the fraction of patients with the disease who have the finding (sensitivity), and the fraction of patients without the disease who have the finding (1-specificity).

In 2000, diagnosis systems using unstructured knowledge started to appear. These systems use some structuring of knowledge such as, for example, entities such as findings and disorders being tagged in documents to facilitate retrieval. ISABEL, for example, uses Autonomy information retrieval software and a database of medical textbooks to retrieve appropriate diagnoses given input findings. Autonomy Auminence uses the Autonomy technology to retrieve diagnoses given findings and organizes the diagnoses by body system. First CONSULT allows one to search a large collection of medical books, journals, and guidelines by chief complaints and age group to arrive at possible diagnoses. PEPID DDX is a diagnosis generator based on PEPID's independent clinical content.

Clinical decision rules have been developed for a number of medical disorders, and computer systems have been developed to help practitioners and patients apply these rules. The Acute Cardiac Ischemia Time-Insensitive Predictive Instrument (ACI-TIPI) takes clinical and ECG features as input and produces probability of acute cardiac ischemia as output to assist with triage of patients with chest pain or other symptoms suggestive of acute cardiac ischemia. ACI-TIPI is incorporated into many commercial heart monitors/defibrillators. The CaseWalker system uses a four-item questionnaire to diagnose major depressive disorder. The PKC Advisor provides guidance on 98 patient problems such as abdominal pain and vomiting.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided, in a data processing system, comprising at least one processor and at least one memory, the at least one memory comprising instructions executed by the at least one processor to cause the at least one processor to implement a cognitive artificial intelligence training mechanism for simulating patients for developing artificial intelligence based medical solutions. The method comprises perturbing, by a non-image based information perturbation engine of the cognitive artificial intelligence training mechanism, non-image based information of a real patient from a real patient data set forming perturbed non-image based information. The method also comprises generating, by an artificial patient assembly engine of the cognitive artificial intelligence training mechanism, an artificial patient data in an artificial patient data set using the perturbed non-image based information and a non-perturbed medical image of the real patient. Additionally, the method comprises training, by a training engine of the cognitive artificial intelligence training mechanism, an operation of a learning algorithm utilized by the cognitive data processing system using real patient data in the real patient data set and the artificial patient data in the artificial patient data set.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 depicts a schematic diagram of one illustrative embodiment of a cognitive system in a computer network;

FIG. 2 is a block diagram of an example data processing system in which aspects of the illustrative embodiments are implemented;

FIG. 3 is an example diagram illustrating an interaction of elements of a cognitive system in accordance with one illustrative embodiment; and

FIG. 4 depicts an exemplary flowchart of the operation performed by a cognitive system in implementing a cognitive artificial intelligence training mechanism that simulates patients for developing artificial intelligence based medical solutions within a data processing system in accordance with an illustrative embodiment.

### DETAILED DESCRIPTION

Machine learning-based algorithms, especially deep-learning algorithms, for medical applications require datasets of a large number of patients with specific conditions to ensure that a model will maintain its performance on unseen patient populations. For supervised learning, establishing a ground truth, e.g., whether a patient has a particular condition or disease or not, whether a lesion is circular or has irregular shape, etc., is also essential. For unsupervised learning, while the ground truth is not required, usually the number of patients required to achieve a desired performance is even higher. For solutions based on medical images, acquisition of the imaging studies by different vendors is also essential. To ensure generalizability for a machine learning-based solution, besides the ground truth, patients in the training dataset should cover variability on various patient characteristics, e.g., race, gender, age, medical history, family history, clinical risk factors, co-morbidities, different levels of a certain condition, or the like.

Obtaining sufficiently large datasets for training artificial intelligence based systems is a significant issue. Generating such large datasets tends to be a very resource intensive process requiring large amounts of subject matter expert time to perform. Moreover, such processes tend to be a manual process that is subject to the error prone nature of manual processes. Thus, the illustrative embodiments provide mechanisms for simulating patients for developing artificial intelligence based medical solutions. That is, the mechanisms expand a dataset representing patients for purposes of training an artificial intelligence system that operates on patient information. The mechanisms start with a patient data set of information and medical images of real patients set having an acceptable reliability. In accordance with the present invention, acceptable means that the information and medical images are produced using technology, personnel, and procedures that would be recognized as meeting the standard of care in a typical hospital. The system generates perturbations in the information and the medical image of the real patients in the patient data set to generate a larger patient data set that includes information and medical images for both the real patients and artificial patients that are derived from the real patients. To generate the portion of the patient data set associated with the artificial patients, the mechanisms perturb real patient information to generate artificial patient information, perturb medical images to generate artificial medical images that are consistent with the perturbed patient information, and expand the patient data set with the artificial patient information and associated artificial medical images. The larger patient data set that includes the information for both the real patients and artificial patients that represent real patients is then utilized to train the operation of deep-learning based algorithms to operate more efficiently and accurately than would otherwise be possible and ensure that the deep-learning based algorithms perform per requirements for unseen patient population.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general purpose hardware, a procedure or method for executing the functions, or a combination of any of the above.

The present description and claims may make use of the terms “a,” “at least one of,” and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

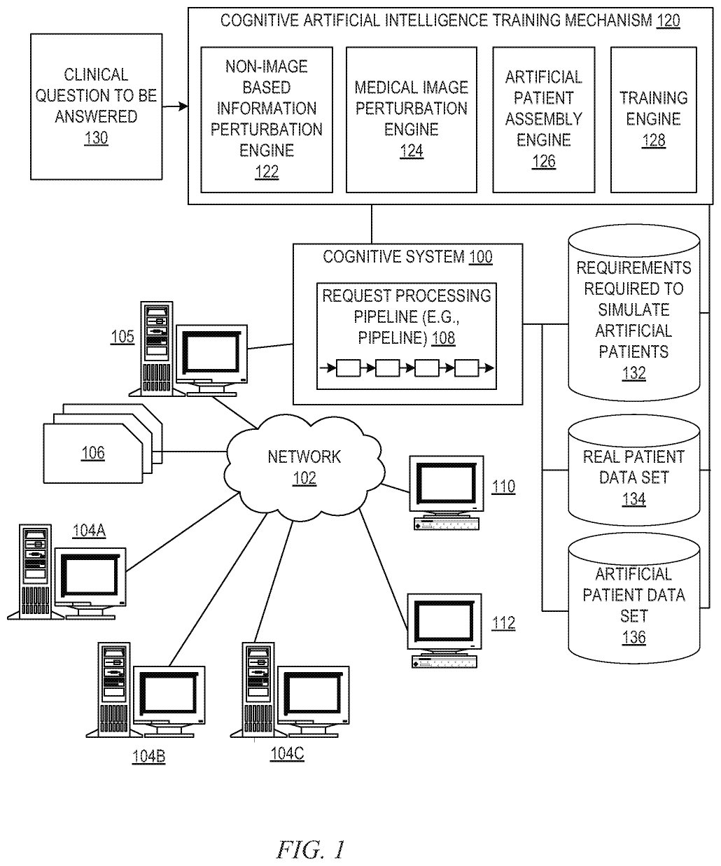

As noted above, the present invention provides mechanisms for simulating patients for developing artificial intelligence based medical solutions. The illustrative embodiments may be utilized in many different types of data processing environments. In order to provide a context for the description of the specific elements and functionality of the illustrative embodiments, FIGS. 1-3 are provided hereafter as example environments in which aspects of the illustrative embodiments may be implemented. It should be appreciated that FIGS. 1-3 are only examples and are not intended to assert or imply any limitation with regard to the environments in which aspects or embodiments of the present invention may be implemented. Many modifications to the depicted environments may be made without departing from the spirit and scope of the present invention.

FIGS. 1-3 are directed to describing an example cognitive system for generating artificial patient information and medical images based on real patient information and medical images for use in training the operation of deep-learning based algorithms to operate more efficiently and accurately than would otherwise be possible and ensure that the deep-learning based algorithms perform per requirements for unseen patient population that implements a request processing pipeline, request processing methodology, and request processing computer program product with which the mechanisms of the illustrative embodiments are implemented. These requests may be provided as structure or unstructured request messages, natural language questions, or any other suitable format for requesting an operation to be performed by the cognitive system. As described in more detail hereafter, the particular application that is implemented in the cognitive system of the present invention is an application for simulating patients for developing artificial intelligence based medical solutions.

It should be appreciated that the cognitive system, while shown as having a single request processing pipeline in the examples hereafter, may in fact have multiple request processing pipelines. Each request processing pipeline may be separately trained and/or configured to process requests associated with different domains or be configured to perform the same or different analysis on input requests, depending on the desired implementation. For example, in some cases, a first request processing pipeline may be trained to operate on input requests directed to a generating a probability of malignancy of a detected lung nodule. In other cases, for example, the request processing pipelines may be configured to provide different types of cognitive functions or support different types of applications, such as one request processing pipeline being used for generating a probability of malignancy within breast tissue, etc.

Moreover, each request processing pipeline may have its own associated corpus or corpora that they ingest and operate on, e.g., one corpus for lung cancer domain related documents and another corpus for breast cancer domain related documents in the above examples. In some cases, the request processing pipelines may each operate on the same domain of input questions but may have different configurations, e.g., different annotators or differently trained annotators, such that different analysis and potential responses are generated. The cognitive system may provide additional logic for routing requests to the appropriate request processing pipeline, such as based on a determined domain of the input request, combining and evaluating final results generated by the processing performed by multiple request processing pipelines, and other control and interaction logic that facilitates the utilization of multiple request processing pipelines.

It should be appreciated that while the present invention will be described in the context of the cognitive system implementing one or more request processing pipelines that operate on a request, the illustrative embodiments are not limited to such. Rather, the mechanisms of the illustrative embodiments may operate on requests that are posed as “questions” or formatted as requests for the cognitive system to perform cognitive operations on a specified set of input data using the associated corpus or corpora and the specific configuration information used to configure the cognitive system.

As will be discussed in greater detail hereafter, the illustrative embodiments may be integrated in, augment, and extend the functionality of these pipelines, or request processing pipeline, mechanisms of a healthcare cognitive system with regard to simulating patients for developing artificial intelligence based medical solutions. For example, identifying an initial patient data set which is diverse enough with regard to patient features (ethnicity, age, level of emphysema, other conditions, or the like) to represent seed data set that may be used to generate a much larger data set. For each of these diverse features, the cognitive system perturbs not only the diverse features but also one or more medical images associated with the initial patient data set to generate new medical images for artificially generated patients, but which correlate with the perturbations made to other diverse features of the artificially generated patients, i.e. the perturbations are not random and instead a consistent perturbation is generated. For example, medical images may be perturbed but these perturbations may be focused on areas that are consistent with a change in a level of emphysema made by other perturbations for generating this artificial patient. Utilizing the perturbed patient features and medical images for the artificial patients as well as the features and medical images of the real patients, the cognitive system trains the operation of deep-learning based algorithms to operate more efficiently and accurately than would otherwise be possible and ensure that the deep-learning based algorithms perform per requirements for unseen patient population.

It should be appreciated that the mechanisms described in FIGS. 1-3 are only examples and are not intended to state or imply any limitation with regard to the type of cognitive system mechanisms with which the illustrative embodiments are implemented. Many modifications to the example cognitive system shown in FIGS. 1-3 may be implemented in various embodiments of the present invention without departing from the spirit and scope of the present invention.

As an overview, a cognitive system is a specialized computer system, or set of computer systems, configured with hardware and/or software logic (in combination with hardware logic upon which the software executes) to emulate human cognitive functions. These cognitive systems apply human-like characteristics to conveying and manipulating ideas which, when combined with the inherent strengths of digital computing, can solve problems with high accuracy and resilience on a large scale. A cognitive system performs one or more computer-implemented cognitive operations that approximate a human thought process as well as enable people and machines to interact in a more natural manner so as to extend and magnify human expertise and cognition. A cognitive system comprises artificial intelligence logic, such as natural language processing (NLP) based logic, for example, and machine learning logic, which may be provided as specialized hardware, software executed on hardware, or any combination of specialized hardware and software executed on hardware. The logic of the cognitive system implements the cognitive operation(s), examples of which include, but are not limited to, question answering, identification of related concepts within different portions of content in a corpus, intelligent search algorithms, such as Internet web page searches, for example, medical diagnostic and treatment recommendations, and other types of recommendation generation, e.g., items of interest to a particular user, potential new contact recommendations, or the like.

IBM Watson™ is an example of one such cognitive system which can process human readable language and identify inferences between text passages with human-like high accuracy at speeds far faster than human beings and on a larger scale. In general, such cognitive systems are able to perform the following functions:
Navigate the complexities of human language and understanding,
Ingest and process vast amounts of structured and unstructured data,
Generate and evaluate hypothesis,
Weigh and evaluate responses that are based only on relevant evidence,
Provide situation-specific advice, insights, and guidance,
Improve knowledge and learn with each iteration and interaction through machine learning processes,
Enable decision making at the point of impact (contextual guidance),
Scale in proportion to the task,
Extend and magnify human expertise and cognition,
Identify resonating, human-like attributes and traits from natural language,
Deduce various language specific or agnostic attributes from natural language,
High degree of relevant recollection from data points (images, text, voice) (memorization and recall),
Predict and sense with situational awareness that mimic human cognition based on experiences, or
Answer questions based on natural language and specific evidence.

In one aspect, cognitive systems provide mechanisms for responding to requests posed to these cognitive systems using a request processing pipeline and/or process requests which may or may not be posed as natural language requests. The requests processing pipeline is an artificial intelligence application executing on data processing hardware that responds to requests pertaining to a given subject-matter domain presented in natural language. The request processing pipeline receives inputs from various sources including input over a network, a corpus of electronic documents or other data, data from a content creator, information from one or more content users, and other such inputs from other possible sources of input. Data storage devices store the corpus of data. A content creator creates content in a document for use as part of a corpus of data with the request processing pipeline. The document may include any file, text, article, or source of data for use in the requests processing system. For example, a request processing pipeline accesses a body of knowledge about the domain, or subject matter area, e.g., financial domain, medical domain, legal domain, etc., where the body of knowledge (knowledgebase) can be organized in a variety of configurations, e.g., a structured repository of domain-specific information, such as ontologies, or unstructured data related to the domain, or a collection of natural language documents about the domain.

Content users input requests to cognitive system which implements the request processing pipeline. The request processing pipeline then responds to the requests using the content in the corpus of data by evaluating documents, sections of documents, portions of data in the corpus, or the like. When a process evaluates a given section of a document for semantic content, the process can use a variety of conventions to query such document from the request processing pipeline, e.g., sending the query to the request processing pipeline as a well-formed requests which is then interpreted by the request processing pipeline and a response is provided containing one or more responses to the request. Semantic content is content based on the relation between signifiers, such as words, phrases, signs, and symbols, and what they stand for, their denotation, or connotation. In other words, semantic content is content that interprets an expression, such as by using Natural Language Processing.

As will be described in greater detail hereafter, the request processing pipeline receives a request, parses the request to extract the major features of the request, uses the extracted features to formulate queries, and then applies those queries to the corpus of data. Based on the application of the queries to the corpus of data, the request processing pipeline generates a set of responses to the request, by looking across the corpus of data for portions of the corpus of data that have some potential for containing a valuable response to the request. The request processing pipeline then performs deep analysis on the language of the request and the language used in each of the portions of the corpus of data found during the application of the queries using a variety of reasoning algorithms. There may be hundreds or even thousands of reasoning algorithms applied, each of which performs different analysis, e.g., comparisons, natural language analysis, lexical analysis, or the like, and generates a score. For example, some reasoning algorithms may look at the matching of terms and synonyms within the language of the request and the found portions of the corpus of data. Other reasoning algorithms may look at temporal or spatial features in the language, while others may evaluate the source of the portion of the corpus of data and evaluate its veracity.

As mentioned above, request processing pipeline mechanisms operate by accessing information from a corpus of data or information (also referred to as a corpus of content), analyzing it, and then generating answer results based on the analysis of this data. Accessing information from a corpus of data typically includes: a database query that answers requests about what is in a collection of structured records, and a search that delivers a collection of document links in response to a query against a collection of unstructured data (text, markup language, etc.). Conventional request processing systems are capable of generating answers based on the corpus of data and the input request, verifying answers to a collection of request for the corpus of data, correcting errors in digital text using a corpus of data, and selecting responses to requests from a pool of potential answers, i.e. candidate answers.

FIG. 1 depicts a schematic diagram of one illustrative embodiment of a cognitive system 100 implementing a request processing pipeline 108, which in some embodiments may be a request processing pipeline, in a computer network 102. For purposes of the present description, it will be assumed that the request processing pipeline 108 that operates on structured and/or unstructured requests in the form of requests. One example of a question processing operation which may be used in conjunction with the principles described herein is described in U.S. Patent Application Publication No. 2011/0125734, which is herein incorporated by reference in its entirety. The cognitive system 100 is implemented on one or more computing devices 104A-C (comprising one or more processors and one or more memories, and potentially any other computing device elements generally known in the art including buses, storage devices, communication interfaces, and the like) connected to the computer network 102. For purposes of illustration only, FIG. 1 depicts the cognitive system 100 being implemented on computing device 104A only, but as noted above the cognitive system 100 may be distributed across multiple computing devices, such as a plurality of computing devices 104A-C. The network 102 includes multiple computing devices 104A-C, which may operate as server computing devices, and 110 and 112 which may operate as client computing devices, in communication with each other and with other devices or components via one or more wired and/or wireless data communication links, where each communication link comprises one or more of wires, routers, switches, transmitters, receivers, or the like. In some illustrative embodiments, the cognitive system 100 and network 102 enables request processing functionality for one or more cognitive system users via their respective computing devices 110 and 112. In other embodiments, the cognitive system 100 and network 102 may provide other types of cognitive operations including, but not limited to, request processing and cognitive response generation which may take many different forms depending upon the desired implementation, e.g., cognitive information retrieval, training/instruction of users, cognitive evaluation of data, or the like. Other embodiments of the cognitive system 100 may be used with components, systems, sub-systems, and/or devices other than those that are depicted herein.

The cognitive system 100 is configured to implement a request processing pipeline 108 that receive inputs from various sources. The requests may be posed in the form of natural language request for information, natural language request for the performance of a cognitive operation, or the like. For example, the cognitive system 100 receives input from the network 102, a corpus or corpora of electronic documents 132, 134, and 136, cognitive system users, and/or other data and other possible sources of input. In one embodiment, some or all of the inputs to the cognitive system 100 are routed through the network 102. The various computing devices 104A-D on the network 102 include access points for content creators and cognitive system users. Some of the computing devices 104A-C includes devices for a database storing the corpus or corpora of data 132, 134, and 136 (which is shown as a separate entity in FIG. 1 for illustrative purposes only). Portions of the corpus or corpora of data 132, 134, and 136 may also be provided on one or more other network attached storage devices, in one or more databases, or other computing devices not explicitly shown in FIG. 1. The network 102 includes local network connections and remote connections in various embodiments, such that the cognitive system 100 may operate in environments of any size, including local and global, e.g., the Internet.

In one embodiment, the content creator creates content in a document of the corpus or corpora of data 132, 134, and 136 for use as part of a corpus of data with the cognitive system 100. The document includes any file, text, article, or source of data for use in the cognitive system 100. Cognitive system users access the cognitive system 100 via a network connection or an Internet connection to the network 102, and requests to the cognitive system 132, 134, and 136 that are responded to/processed based on the content in the corpus or corpora of data 132, 134, and 136. In one embodiment, the requests are formed using natural language. The cognitive system 100 parses and interprets the request via a pipeline 108, and provides a response to the cognitive system user, e.g., cognitive system user 110, containing one or more responses to the request posed, response to the request, results of processing the request, or the like. In some embodiments, the cognitive system 100 provides a response to users in a ranked list of candidate responses while in other illustrative embodiments, the cognitive system 100 provides a single final response or a combination of a final response and ranked listing of other candidate responses.

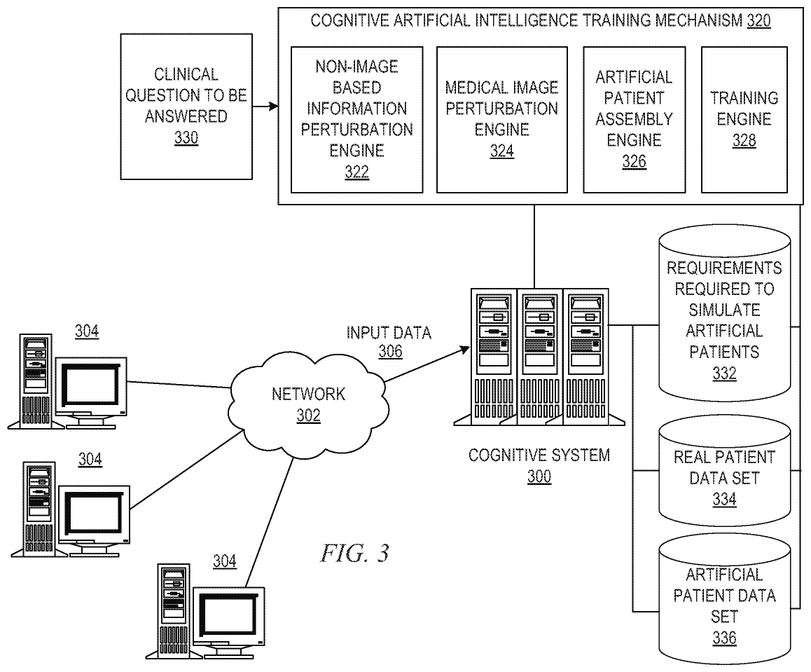

The cognitive system 100 implements the pipeline 108 which comprises a plurality of stages for processing a request based on information obtained from the corpus or corpora of data 132, 134, and 136. The pipeline 108 generates responses for the request based on the processing of the request and the corpus or corpora of data 132, 134, and 136. The pipeline 108 will be described in greater detail hereafter with regard to FIG. 3.

In some illustrative embodiments, the cognitive system 100 may be the IBM Watson™ cognitive system available from International Business Machines Corporation of Armonk, N.Y., which is augmented with the mechanisms of the illustrative embodiments described hereafter. As outlined previously, a pipeline of the IBM Watson™ cognitive system receives a request which it then parses to extract the major features of the request, which in turn are then used to formulate queries that are applied to the corpus or corpora of data 132, 134, and 136.

The scores obtained from the various reasoning algorithms are then weighted against a statistical model that summarizes a level of confidence that the pipeline 108 of the IBM Watson™ cognitive system 100, in this example, has regarding the evidence that the potential candidate response is inferred by the request. This process is be repeated for each of the candidate responses to generate ranked listing of candidate responses which may then be presented to the user that submitted the request, e.g., a user of client computing device 110, or from which a final response is selected and presented to the user. More information about the pipeline 108 of the IBM Watson™ cognitive system 100 may be obtained, for example, from the IBM Corporation website, IBM Redbooks, and the like. For example, information about the pipeline of the IBM Watson™ cognitive system can be found in Yuan et al., “Watson and Healthcare,” IBM developerWorks, 2011 and “The Era of Cognitive Systems: An Inside Look at IBM Watson and How it Works” by Rob High, IBM Redbooks, 2012.

As noted above, while the input to the cognitive system 100 from a client device may be posed in the form of a natural language question, the illustrative embodiments are not limited to such. Rather, the input question may in fact be formatted or structured as any suitable type of request which may be parsed and analyzed using structured and/or unstructured input analysis, including but not limited to the natural language parsing and analysis mechanisms of a cognitive system such as IBM Watson™, to determine the basis upon which to perform cognitive analysis and providing a result of the cognitive analysis. In the case of a healthcare based cognitive system, this analysis may involve processing patient medical records, medical guidance documentation from one or more corpora, and the like, to provide a healthcare oriented cognitive system result.

In the context of the present invention, cognitive system 100 may provide a cognitive functionality for simulating patients for developing artificial intelligence based medical solutions. For example, depending upon the particular implementation, the knowledge base expansion based operations may comprise artificial intelligence system training, patient diagnostics, medical treatment recommendation systems, medical practice management systems, personal patient care plan generation and monitoring, patient electronic medical record (EMR) evaluation for various purposes, such as for identifying patients that are suitable for a medical trial or a particular type of medical treatment, or the like. Thus, the cognitive system 100 may be a healthcare cognitive system 100 that operates in the medical or healthcare type domains and which may process requests for such healthcare operations via the request processing pipeline 108 input as either structured or unstructured requests, natural language input questions, or the like. In one illustrative embodiment, the cognitive system 100 is a cognitive artificial intelligence training system that simulates patients for training the operation of deep-learning based algorithms to operate more efficiently and accurately than would otherwise be possible and ensure that the deep-learning based algorithms perform per requirements for unseen patient population.

As shown in FIG. 1, the cognitive system 100 is further augmented, in accordance with the mechanisms of the illustrative embodiments, to include logic implemented in specialized hardware, software executed on hardware, or any combination of specialized hardware and software executed on hardware, for implementing cognitive artificial intelligence training mechanism 120 that generates perturbations in a small patient data set associated with real patients to generate artificial patient data that represents realistic patients and increase the patient data set. Cognitive artificial intelligence training mechanism 120 considers/represents a patient from a holistic point of view including non-image based information, such as demographics, family and medical history, lab results, radiology and other reports, or the like, as well as medical images that may manifest a particular medical malady. Thus, cognitive artificial intelligence training mechanism 120 perturbs non-image based information and medical images of real patients so as to generate artificial patient information and medical images in order to expand the patient data set. Both the non-image based information and medical images associated with the real patients and the perturbed non-image based information and medical images associated with the artificial patients are then utilized to train the operation of deep-learning based algorithms to operate more efficiently and accurately than would otherwise be possible and ensure that the deep-learning based algorithms perform per requirements for unseen patient population. Accordingly, as shown in FIG. 1, cognitive artificial intelligence training mechanism 120 comprises non-image based information perturbation engine 122, medical image perturbation engine 124, artificial patient assembly engine 126, and training engine 128.

Cognitive artificial intelligence training mechanism 120 operates based on an expectation of a clinical question to be answered 130, such as a future medical professional posing an inquiry as to whether a particular patient might have a particular medical malady, such as lung cancer, breast cancer, congestive heart failure, polyps, or the like. The following utilizes lung cancer as one example of the operation of cognitive artificial intelligence training mechanism 120, but the illustrative embodiments are not limited to only this medical malady. As stated previously, cognitive system 100 may be an artificial intelligence system that may not have enough patient data to accurately provide a positive or negative indication of the exemplary lung cancer. Therefore, prior to processing a clinical question to be answered 130, non-image based information perturbation engine 122 identifies requirements required to simulate artificial patients 132 as well as a patient data set associated with real patients 134 who have been tested for lung cancer and have had either a positive or negative lung cancer diagnosis. That is, a same clinical question that a medical professional is seeking an answer to is used for training cognitive system 100 and, once trained, cognitive system 100 answers the same clinical question of a “future” unseen patient.

Utilizing the requirements required to simulate artificial patients 132 and the non-image based information associated with the real patients from the patient data set associated with real patients 134, in one embodiment, non-image based information perturbation engine 122 perturbs the non-image based information in order to generate non-image based information for an artificial patient. For example, non-image based information perturbation engine 122 makes one or more changes to structured and/or non-structured non-image based information, such as smoking history, other diseases, family history, age, body mass index, medical history, lab results, radiology and other reports, or the like to generate perturbed non-image based information for one or more artificial patients. For each real patient for whose non-image based information has been perturbed by non-image based information perturbation engine 122, medical image perturbation engine 124 then utilizes one or more associated medical images from the patient data set associated with real patients 134 and associated perturbed non-image based information to perturb the one or more associated medical images and generate one or more perturbed medical images.

In another embodiment, medical image perturbation engine 124 perturbs one or more medical images from the patient data set associated with real patients 134 to generate one or more perturbed medical images prior to the non-image based information perturbation engine 122 perturbs the non-image based information associated with the real patients from the patient data set associated with real patients 134 to generate non-image based information for an artificial patient. In yet another embodiment, medical image perturbation engine 124 perturbs one or more medical images from the patient data set associated with real patients 134 at substantially a same time as non-image based information perturbation engine 122 perturbs the non-image based information associated with the real patients from the patient data set associated with real patients 134 to generate one or more perturbed medical images and associated non-image based information for an artificial patient, respectively. In still another embodiment, non-image based information perturbation engine 122 perturbs non-image based information but an actual medical image is utilized from a real patient in order to generate non-image based information for an artificial patient. That is, for example, electronic medical records of a real patient says that no family history of lung cancer, but non-image based information perturbation engine 122 generates an artificial patient using the electronic medical records of the real patient such that the patient's father has lung cancer associated with the non-perturbed medical image of the real patient.

The perturbations performed by non-image based information perturbation engine 122 and medical image perturbation engine 124 may operate on patient data of both positively diagnosed patients and negatively diagnosed patients. That is, medical image perturbation engine 124 may copy of malignant lung nodules or masses from a medical image of a positive patient and paste them into an image from a negative patient, to make the image appear as if it is from a positive patient. This could conceivably also include non-image based information perturbation engine 122 changing of the text in an accompanying radiology report from a state in which it says there are no nodules found, to a new state in which it notes the presence and location of the nodule that was pasted. The same can be done for breast lesion for breast cancer, or liver cancer or colon cancer using different imaging modalities, such as computerized axial tomography (CT) scan, magnetic resonance imaging (MRI) scan, ultrasound (U/S) scan, positron emission tomography (PET) scan, X-ray scan, or the like. A reverse operation may be performed for a positive patient where lung nodules or massed are removed from medical image and the text in an accompanying radiology report is changed from a state in which it says there are nodules found, to a new state in which it notes the absence of any nodules.

Thus, the goal of non-image based information perturbation engine 122 and medical image perturbation engine 124 is to take non-image based information and medical images of real patients to generate new artificial patients with non-image based information and medical images that are realistic enough to be used in a subsequent machine learning endeavor to train the operation of deep-learning based algorithms to discriminate between the two kinds of patients. Accordingly, for a particular artificial patient, artificial patient assembly engine 126 utilizes the perturbed non-image based information perturbed by non-image based information perturbation engine 122 and the associated perturbed one or more medical images perturbed by medical image perturbation engine 124 to assemble a patient data set associated with artificial patients 136.

Thus, artificial patient assembly engine 126 generates artificial patient data 136 for artificial patients that represent, for each artificial patient, being either positively diagnosed for lung cancer or negatively diagnosed for lung cancer. Utilizing the patient data set associated with artificial patients 136, both positively and negatively diagnosed, and the patient data set associated with real patients 134, both positively and negatively diagnosed, training engine 128 trains the operation of deep-learning based algorithms executed by cognitive system 100 to discriminate between the two kinds of patients as well as, optionally, between real patients and artificial patients. Deep-learning based algorithms are only one example of machine learning. The patient data set associated with artificial patients 136 and patient data set associated with real patients 134 may be also used to train any machine learning based system, e.g. different classifiers, such as neural networks, support vector machines, decision trees, ensemble of classifiers (e.g. random forests), etc., without departing from the spirit and scope of the invention. Training engine 128 takes in either one of the images from the patient data set associated with artificial patients 136 or else one of the images from the patient data set associated with real patients 134, and tries to predict whether the image is: 1) real positive non-image based information and medical image; 2) real negative non-image based information and medical image; 3) artificial positive non-image based information and medical image, or 4) artificial negative non-image based information and medical image.

Utilizing a specialized adversarial loss function, training engine 128 quantifies the extent to which this goal is achieved and drives backpropagation updates to non-image based information perturbation engine 122, medical image perturbation engine 124, and artificial patient assembly engine 126. That is, in one embodiment, training engine 128 adjusts weights associated with the non-image based information changes and the medical image changes made by non-image based information perturbation engine 122 and medical image perturbation engine 124, respectively, so that future changes will provide more accurate non-image based information changes and the medical image changes thereby increasing the efficiency and accuracy of the operation of the deep-learning based algorithms. Therefore, training engine 128 backpropagation adjusts the weights so that non-image based information perturbation engine 122 and medical image perturbation engine 124 produces non-image based information and medical images that are more likely to be used in the training of the deep-learning based algorithms. The illustrative embodiments recognize that training engine 128 may quantify the extent to which a goal is achieved and drive backpropagation updates to non-image based information perturbation engine 122, medical image perturbation engine 124, and artificial patient assembly engine 126 in other manners, without departing from the spirit and scope of the present invention.

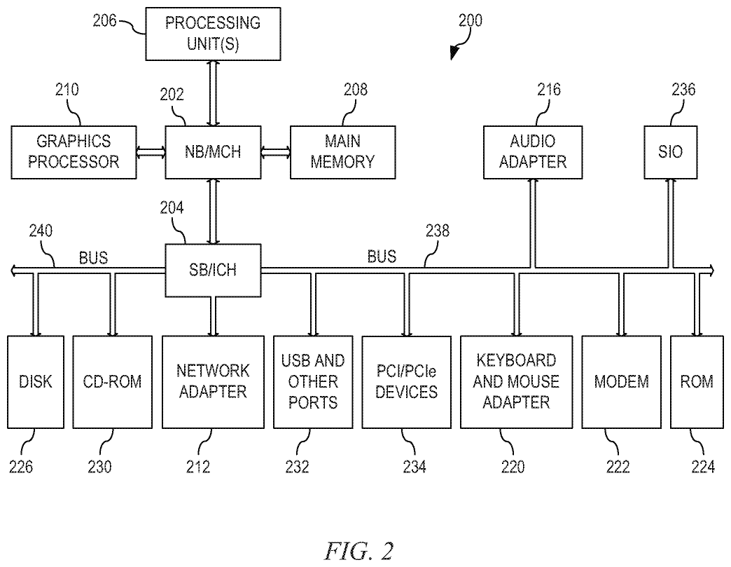

As noted above, the mechanisms of the illustrative embodiments are rooted in the computer technology arts and are implemented using logic present in such computing or data processing systems. These computing or data processing systems are specifically configured, either through hardware, software, or a combination of hardware and software, to implement the various operations described above. As such, FIG. 2 is provided as an example of one type of data processing system in which aspects of the present invention may be implemented. Many other types of data processing systems may be likewise configured to specifically implement the mechanisms of the illustrative embodiments.

FIG. 2 is a block diagram of an example data processing system in which aspects of the illustrative embodiments are implemented. Data processing system 200 is an example of a computer, such as server 104 or client 110 in FIG. 1, in which computer usable code or instructions implementing the processes for illustrative embodiments of the present invention are located. In one illustrative embodiment, FIG. 2 represents a server computing device, such as a server 104, which, which implements a cognitive system 100 and system pipeline 108 augmented to include the additional mechanisms of the illustrative embodiments described hereafter.

In the depicted example, data processing system 200 employs a hub architecture including north bridge and memory controller hub (NB/MCH) 202 and south bridge and input/output (I/O) controller hub (SB/ICH) 204. Processing unit 206, main memory 208, and graphics processor 210 are connected to NB/MCH 202. Graphics processor 210 is connected to NB/MCH 202 through an accelerated graphics port (AGP).

In the depicted example, local area network (LAN) adapter 212 connects to SB/ICH 204. Audio adapter 216, keyboard and mouse adapter 220, modem 222, read only memory (ROM) 224, hard disk drive (HDD) 226, CD-ROM drive 230, universal serial bus (USB) ports and other communication ports 232, and PCI/PCIe devices 234 connect to SB/ICH 204 through bus 238 and bus 240. PCI/PCIe devices may include, for example, Ethernet adapters, add-in cards, and PC cards for notebook computers. PCI uses a card bus controller, while PCIe does not. ROM 224 may be, for example, a flash basic input/output system (BIOS).

HDD 226 and CD-ROM drive 230 connect to SB/ICH 204 through bus 240. HDD 226 and CD-ROM drive 230 may use, for example, an integrated drive electronics (IDE) or serial advanced technology attachment (SATA) interface. Super I/O (SIO) device 236 is connected to SB/ICH 204.

An operating system runs on processing unit 206. The operating system coordinates and provides control of various components within the data processing system 200 in FIG. 2. As a client, the operating system is a commercially available operating system such as Microsoft® Windows 8®. An object-oriented programming system, such as the Java™ programming system, may run in conjunction with the operating system and provides calls to the operating system from Java™ programs or applications executing on data processing system 200.

As a server, data processing system 200 may be, for example, an IBM® cServer™ System p® computer system, running the Advanced Interactive Executive (AIX®) operating system or the LINUX® operating system. Data processing system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and are loaded into main memory 208 for execution by processing unit 206. The processes for illustrative embodiments of the present invention are performed by processing unit 206 using computer usable program code, which is located in a memory such as, for example, main memory 208, ROM 224, or in one or more peripheral devices 226 and 230, for example.

A bus system, such as bus 238 or bus 240 as shown in FIG. 2, is comprised of one or more buses. Of course, the bus system may be implemented using any type of communication fabric or architecture that provides for a transfer of data between different components or devices attached to the fabric or architecture. A communication unit, such as modem 222 or network adapter 212 of FIG. 2, includes one or more devices used to transmit and receive data. A memory may be, for example, main memory 208, ROM 224, or a cache such as found in NB/MCH 202 in FIG. 2.

Those of ordinary skill in the art will appreciate that the hardware depicted in FIGS. 1 and 2 may vary depending on the implementation. Other internal hardware or peripheral devices, such as flash memory, equivalent non-volatile memory, or optical disk drives and the like, may be used in addition to or in place of the hardware depicted in FIGS. 1 and 2. Also, the processes of the illustrative embodiments may be applied to a multiprocessor data processing system, other than the SMP system mentioned previously, without departing from the spirit and scope of the present invention.

Moreover, the data processing system 200 may take the form of any of a number of different data processing systems including client computing devices, server computing devices, a tablet computer, laptop computer, telephone or other communication device, a personal digital assistant (PDA), or the like. In some illustrative examples, data processing system 200 may be a portable computing device that is configured with flash memory to provide non-volatile memory for storing operating system files and/or user-generated data, for example. Essentially, data processing system 200 may be any known or later developed data processing system without architectural limitation.

FIG. 3 is an example diagram illustrating an interaction of elements of a cognitive system in accordance with one illustrative embodiment. The example diagram of FIG. 3 depicts an implementation of a cognitive system 300, which may be a cognitive system such as cognitive system 100 described in FIG. 1, that is configured to implement simulation of patients for developing artificial intelligence based medical solutions by training the operation of deep-learning based algorithms utilized by cognitive systems, such as cognitive system 100, to operate more efficiently and accurately than would otherwise be possible and ensure that the deep-learning based algorithms perform per requirements for unseen patient population. Again, deep-learning based algorithms are only one example of machine learning. The patient data set associated with artificial patients 136 and patient data set associated with real patients 134 may be also used to train any machine learning based system, e.g. different classifiers, such as neural networks, support vector machines, decision trees, ensemble of classifiers (e.g. random forests), etc., without departing from the spirit and scope of the invention. However, it should be appreciated that this is only an example implementation and other simulation of patients for developing artificial intelligence based medical solutions may be implemented in other embodiments of the cognitive system 100 without departing from the spirit and scope of the present invention.

As is shown in FIG. 3, cognitive system 300 receives input data in the way of a clinical question to be answered 330 from one or more computing device 304 via a network 302. In accordance with the illustrative embodiments herein, cognitive system 300 is augmented to include cognitive artificial intelligence training mechanism 320. Cognitive artificial intelligence training mechanism 320 comprises non-image based information perturbation engine 322, medical image perturbation engine 324, artificial patient assembly engine 326, and training engine 328, which operate in a similar manner as previously described above with regard to corresponding elements 122-128 in FIG. 1.

Cognitive artificial intelligence training mechanism 320 operates based on an expectation of a clinical question to be answered 330, such as a future medical professional posing an inquiry as to whether a particular patient might have a particular medical malady, such as lung cancer, breast cancer, congestive heart failure, polyps, or the like. The following utilizes lung cancer as one example of the operation of cognitive artificial intelligence training mechanism 320, but the illustrative embodiments are not limited to only this medical malady. As stated previously, cognitive system 300 may be an artificial intelligence system that may not have enough patient data to accurately provide a positive or negative indication of lung cancer. Therefore, prior to processing a clinical question to be answered 330, non-image based information perturbation engine 322 identifies requirements required to simulate artificial patients 332 as well as a patient data set associated with real patients 334 who have been tested for lung cancer and have had either a positive or negative lung cancer diagnosis. That is, a same clinical question that a medical professional is seeking an answer to is used for training cognitive system 100 and, once trained, cognitive system 100 answers the same clinical question of a “future” unseen patient.

Utilizing the requirements required to simulate artificial patients 332 and the non-image based information associated with the real patients from the patient data set associated with real patients 334, in one embodiment, non-image based information perturbation engine 322 perturbs the non-image based information in order to generate non-image based information for an artificial patient. For example, non-image based information perturbation engine 322 makes one or more changes to structured and/or non-structured non-image based information, such as smoking history, other diseases, family history, age, body mass index, medical history, lab results, radiology and other reports, or the like to generate perturbed non-image based information for one or more artificial patients. For each real patient for whose non-image based information has been perturbed by non-image based information perturbation engine 322, medical image perturbation engine 324 then utilizes one or more associated medical images from the patient data set associated with real patients 334 and associated perturbed non-image based information to perturb the one or more associated medical images and generate one or more perturbed medical images.

In another embodiment, medical image perturbation engine 324 perturbs one or more medical images from the patient data set associated with real patients 334 to generate one or more perturbed medical images prior to the non-image based information perturbation engine 322 perturbs the non-image based information associated with the real patients from the patient data set associated with real patients 334 to generate non-image based information for an artificial patient. In yet another embodiment, medical image perturbation engine 324 perturbs one or more medical images from the patient data set associated with real patients 334 at substantially a same time as non-image based information perturbation engine 322 perturbs the non-image based information associated with the real patients from the patient data set associated with real patients 334 to generate one or more perturbed medical images and associated non-image based information for an artificial patient, respectively. In still another embodiment, non-image based information perturbation engine 122 perturbs non-image based information but an actual medical image is utilized from a real patient in order to generate non-image based information for an artificial patient. That is, for example, electronic medical records of a real patient says that no family history of lung cancer, but non-image based information perturbation engine 122 generates an artificial patient using the electronic medical records of the real patient such that the patient's father has lung cancer associated with the non-perturbed medical image of the real patient.

The perturbations performed by medical image perturbation engine 324 may operate on patient data of both positively diagnosed patients and negatively diagnosed patients. That is, non-image based information perturbation engine 322 and medical image perturbation engine 324 may copy of malignant lung nodules or masses from a medical image of a positive patient and paste them into an image from a negative patient, to make the image appear as if it is from a positive patient. This could conceivably also include non-image based information engine 322 the changing of the text in an accompanying radiology report from a state in which it says there are no nodules found, to a new state in which it notes the presence and location of the nodule that we pasted. The same can be done for breast lesion for breast cancer, or liver cancer or colon cancer using different imaging modalities, such as computerized axial tomography (CT) scan, magnetic resonance imaging (MRI) scan, ultrasound (U/S) scan, positron emission tomography (PET) scan, X-ray scan, or the like. A reverse operation may be performed for a positive patient where lung nodules or masses are removed from medical image and the text in an accompanying radiology report is changed from a state in which it says there are nodules found, to a new state in which it notes the absence of any nodules.

Thus, the goal of non-image based information perturbation engine 322 and medical image perturbation engine 324 is to take non-image based information and medical images of real patient to generate new artificial patients with non-image based information and medical images that are realistic enough to be used in a subsequent machine learning endeavor to train the operation of deep-learning based algorithms to discriminate between the two kinds of patients. Deep-learning based algorithms are only one example of machine learning. The patient data set associated with artificial patients 136 and patient data set associated with real patients 134 may be also used to train any machine learning based system, e.g. different classifiers, such as neural networks, support vector machines, decision trees, ensemble of classifiers (e.g. random forests), etc., without departing from the spirit and scope of the invention. Accordingly, for a particular real patient, artificial patient assembly engine 326 utilizes the perturbed non-image based information perturbed by non-image based information perturbation engine 322 and the associated perturbed one or more medical images perturbed by medical image perturbation engine 324 to assemble a patient data set associated with artificial patients 336.

Thus, artificial patient assembly engine 326 generates artificial patient data 336 for artificial patients that represent, for each artificial patient, being either positively diagnosed for lung cancer or negatively diagnosed for lung cancer. Utilizing the patient data set associated with artificial patients 336, both positively and negatively diagnosed, and the patient data set associated with real patients 334, both positively and negatively diagnosed, training engine 328 trains the operation of algorithms executed by cognitive system 300 to discriminate between the two kinds of patients as well as, optionally, between real patients and artificial patients. Training engine 328 takes in either one of the images from the patient data set associated with artificial patients 336 or else one of the images from the patient data set associated with real patients 334, and tries to predict whether the image is: 1) real positive non-image based information and medical image; 2) real negative non-image based information and medical image; 3) artificial positive non-image based information and medical image, or 4) artificial negative non-image based information and medical image.

Utilizing a specialized adversarial loss function, training engine 328 quantifies the extent to which this goal is achieved and drives backpropagation updates to non-image based information perturbation engine 322, medical image perturbation engine 324, and artificial patient assembly engine 326. That is, in one embodiment, training engine 328 adjusts weights associated with the non-image based information changes and the medical image changes made by non-image based information perturbation engine 322 and medical image perturbation engine 324, respectively, so that future changes will provide more accurate non-image based information changes and the medical image changes thereby increasing the efficiency and accuracy of the operation of the deep-learning based algorithms. Therefore, training engine 328 backpropagation adjusts the weights so that non-image based information perturbation engine 322 and medical image perturbation engine 324 produces non-image based information and medical images that are more likely to be used in the training of the deep-learning based algorithms. The illustrative embodiments recognize that training engine 328 may quantify the extent to which a goal is achieved and drive backpropagation updates to non-image based information perturbation engine 322, medical image perturbation engine 324, and artificial patient assembly engine 326 in other manners, without departing from the spirit and scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

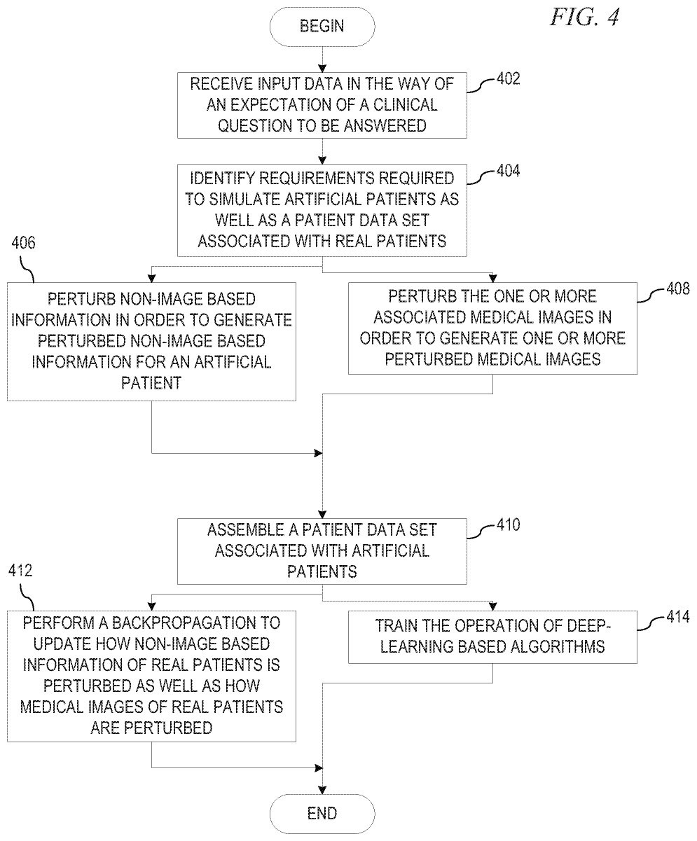

FIG. 4 depicts an exemplary flowchart of the operation performed by a cognitive system in implementing a cognitive artificial intelligence training mechanism that simulates patients for developing artificial intelligence based medical solutions within a data processing system in accordance with an illustrative embodiment. As the operation begins, the cognitive artificial intelligence training mechanism receives input data in the way of an expectation of a clinical question to be answered (step 402). The cognitive artificial intelligence training mechanism identifies requirements required to simulate artificial patients as well as a patient data set associated with real patients who have been tested for lung cancer and have had either a positive or negative lung cancer diagnosis (step 404).

Utilizing the requirements required for simulating artificial patients and non-image based information associated with the real patients from the patient data set associated with real patients, the cognitive artificial intelligence training mechanism perturbs the non-image based information in order to generate perturbed non-image based information for an artificial patient (step 406). Utilizing the requirements required for simulating artificial patients and one or more associated medical images from the patient data set associated with real patients from the patient data set associated with real patients the cognitive artificial intelligence training mechanism perturbs the one or more associated medical images in order to generate one or more perturbed medical images (step 408). It should be noted that steps 406 and 408 may operate at substantially a same time or such that one step follows the other. That is, if one step were to follow the other, the cognitive artificial intelligence training mechanism may perturb the non-image based information of the real patient utilizing perturbed medical images of the artificial patient to generate perturbed non-image based information for the artificial patient. Alternatively, the cognitive artificial intelligence training mechanism may perturb the one or more associated medical images of the real patient utilizing the perturbed non-image based information of the artificial patient to generate one or more perturbed medical images for the artificial patient.

The cognitive artificial intelligence training mechanism then utilizes the perturbed non-image based information perturbed by non-image based information perturbation engine and the associated perturbed one or more medical images perturbed by medical image perturbation engine to assemble a patient data set associated with artificial patients (step 410). From this point, the cognitive artificial intelligence training mechanism may perform a backpropagation to update how non-image based information of real patients is perturbed as well as how medical images of real patients are perturbed (step 412) so that future changes will provide more accurate non-image based information changes and the medical image changes. Additionally, the cognitive artificial intelligence training mechanism utilizes both the real patient data set and the generated artificial patient data set to train the operation of deep-learning based algorithms (step 414) to operate more efficiently and accurately than would otherwise be possible and ensure that the deep-learning based algorithms perform per requirements for unseen patient population. The operation terminates thereafter.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Thus, the illustrative embodiments provide mechanisms for simulating patients for developing artificial intelligence based medical solutions. The mechanisms expand a dataset representing patients for purposes of training an artificial intelligence system that operates on patient information. The mechanisms start with a patient data set of information and medical images of real patients set having an acceptable reliability. The system generates perturbations in the information and the medical image of the real patients in the patient data set to generate a larger patient data set that includes information and medical images for both the real patients and artificial patients that are derived from the real patients. To generate the portion of the patient data set associated with the artificial patients, the mechanisms perturb real patient information to generate artificial patient information, perturb medical images to generate artificial medical images that are consistent with the perturbed patient information, and expand the patient data set with the artificial patient information and associated artificial medical images. The larger patient data set that includes the information for both the real patients and artificial patients that represent the real patients is then utilized to train the operation of deep-learning based algorithms to operate more efficiently and accurately than would otherwise be possible and ensure that the deep-learning based algorithms perform per requirements for unseen patient population.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

1-20. (canceled)

21. A method, in a cognitive data processing system comprising at least one processor and at least one memory, the at least one memory comprising instructions executed by the at least one processor to cause the at least one processor to implement a cognitive artificial intelligence training mechanism for simulating patients for developing artificial intelligence based medical solutions, wherein the cognitive artificial intelligence training mechanism operates to:
perturbing, by a medical image perturbation engine of the cognitive artificial intelligence training mechanism, a medical image of a real patient from a real patient data set forming a perturbed medical image, wherein the medical image of the real patient from the real patient data set is perturbed using perturbed non-image based information;
generating, by an artificial patient assembly engine of the cognitive artificial intelligence training mechanism, an artificial patient data in an artificial patient data set using the perturbed non-image based information and the perturbed medical image of the real patient; and
training, by a training engine of the cognitive artificial intelligence training mechanism, an operation of a learning algorithm utilized by the cognitive data processing system using real patient data in the real patient data set and the artificial patient data in the artificial patient data set.

22. The method of claim 21, wherein the perturbed non-image based information is generated by:
perturbing, by a non-image based information perturbation engine of the cognitive artificial intelligence training mechanism, non-image based information of the real patient from the real patient data set by changing one or more of a smoking history, other diseases, family history, age, body mass index, medical history, lab results, or radiology and other reports.

23. The method of claim 22, wherein the non-image based information of the real patient from the real patient data set is further perturbed using the perturbed medical image.

24. The method of claim 22, wherein the perturbing of the non-image based information of the real patient and the perturbing of the medical image of the real patient is based on a set of requirements required to simulate artificial patients.

25. The method of claim 21, wherein the real patient data set comprises real patients who have been tested for a medical malady and have has either a positive diagnosis of the medical malady or a negative diagnosis for the medical malady.

26. The method of claim 22, wherein the perturbing of the non-image based information of the real patient and the perturbing of the medical image of the real patient comprises:
modifying the non-image based information and the medical image of the real patient with a negative diagnosis for a medical malady such that the perturbed non-image based information and the perturbed medical image indicates that the patient has a positive diagnosis for the medical malady.

27. The method of claim 22, wherein the perturbing of the perturbing of the non-image based information of the real patient and the perturbing of the medical image of the real patient comprises:
modifying the non-image based information and the medical image of the real patient with a positive diagnosis for a medical malady such that the perturbed non-image based information and the perturbed medical image indicates that the patient has a negative diagnosis for the medical malady.

28. The method of claim 21, further comprising:
backpropagating, by the training engine, updates such that further perturbations to non-image based information and medical images of other real patients is modified to provide increased accuracy of future non-image based information changes and the medical image changes.

29. A computer program product comprising a computer readable storage medium having a computer readable program stored therein, wherein the computer readable program, when executed on a data processing system, causes the data processing system to implement a cognitive artificial intelligence training mechanism for simulating patients for developing artificial intelligence based medical solutions, and further causes the data processing system to:
perturb, by a medical image perturbation engine of the cognitive artificial intelligence training mechanism, a medical image of a real patient from a real patient data set forming a perturbed medical image, wherein the medical image of the real patient from the real patient data set is perturbed using perturbed non-image based information;
generate, by an artificial patient assembly engine of the cognitive artificial intelligence training mechanism, an artificial patient data in an artificial patient data set using the perturbed non-image based information and the perturbed medical image of the real patient; and
train, by a training engine of the cognitive artificial intelligence training mechanism, an operation of a learning algorithm utilized by the cognitive data processing system using real patient data in the real patient data set and the artificial patient data in the artificial patient data set.

30. The computer program product of claim 29, wherein the computer readable program further causes the data processing system to generate the perturbed non-image based information by:
perturbing, by a non-image based information perturbation engine of the cognitive artificial intelligence training mechanism, non-image based information of the real patient from the real patient data set by changing one or more of a smoking history, other diseases, family history, age, body mass index, medical history, lab results, or radiology and other reports.

31. The computer program product of claim 30, wherein the non-image based information of the real patient from the real patient data set is further perturbed using the perturbed medical image.

32. The computer program product of claim 30, wherein the perturbing of the non-image based information of the real patient and the perturbing of the medical image of the real patient is based on a set of requirements required to simulate artificial patients.

33. The computer program product of claim 29, wherein the real patient data set comprises real patients who have been tested for a medical malady and have has either a positive diagnosis of the medical malady or a negative diagnosis for the medical malady.

34. The computer program product of claim 30, wherein the computer readable program to perturb the non-image based information of the real patient and to perturb the medical image of the real patient further causes the data processing system to:
modify the non-image based information and the medical image of the real patient with a negative diagnosis for a medical malady such that the perturbed non-image based information and the perturbed medical image indicates that the patient has a positive diagnosis for the medical malady.

35. The computer program product of claim 30, wherein the computer readable program to perturb the non-image based information of the real patient and to perturb the medical image of the real patient further causes the data processing system to:
modifying the non-image based information and the medical image of the real patient with a positive diagnosis for a medical malady such that the perturbed non-image based information and the perturbed medical image indicates that the patient has a negative diagnosis for the medical malady.

36. The computer program product of claim 29, wherein the computer readable program further causes the data processing system to:
backpropagate, by the training engine, updates such that further perturbations to non-image based information and medical images of other real patients is modified to provide increased accuracy of future non-image based information changes and the medical image changes.

37. An apparatus comprising:
at least one processor; and
at least one memory coupled to the at least one processor, wherein the at least one memory comprises instructions which, when executed by the at least one processor, cause the at least one processor to implement a cognitive artificial intelligence training mechanism for simulating patients for developing artificial intelligence based medical solutions, and further causes the at least one processor to:
perturb, by a medical image perturbation engine of the cognitive artificial intelligence training mechanism, a medical image of a real patient from a real patient data set forming a perturbed medical image, wherein the medical image of the real patient from the real patient data set is perturbed using perturbed non-image based information;
generate, by an artificial patient assembly engine of the cognitive artificial intelligence training mechanism, an artificial patient data in an artificial patient data set using the perturbed non-image based information and the perturbed medical image of the real patient; and
train, by a training engine of the cognitive artificial intelligence training mechanism, an operation of a learning algorithm utilized by the cognitive data processing system using real patient data in the real patient data set and the artificial patient data in the artificial patient data set.

38. The apparatus of claim 37, wherein the instructions further cause the at least one processor to:
perturbing, by a non-image based information perturbation engine of the cognitive artificial intelligence training mechanism, non-image based information of the real patient from the real patient data set by changing one or more of a smoking history, other diseases, family history, age, body mass index, medical history, lab results, or radiology and other reports.

39. The apparatus of claim 38, wherein the non-image based information of the real patient from the real patient data set is further perturbed using the perturbed medical image.

40. The apparatus of claim 38, wherein the perturbing of the non-image based information of the real patient and the perturbing of the medical image of the real patient is based on a set of requirements required to simulate artificial patients.
